(19) United States
(12) Patent Application Publication
(10) Pub. No.: US 2004/0138247 A1
(43) Pub. Date: Jul. 15, 2004

(54) Use of desloratadine for treating allergic and inflammatory conditions

(76) Inventors: Melton B. Affrime, Warren, NJ (US); Christopher R. Banfield, High Bridge, NJ (US); Samir K. Gupta, East Brunswick, NJ (US); Desmond Padhi, Thousand Oaks, CA (US)

Correspondence Address: COVINGTON & BURLING, ATTN: PATENT DOCKETING, 1201 PENNSYLVANIA AVENUE, N.W., WASHINGTON, DC, 20004-2401, US

(21) Appl. No.: 10/743,862

(22) Filed: Dec. 24, 2003

Related U.S. Application Data
(63) Continuation of application No. 10/088,708, filed on Mar. 20, 2002, now abandoned.
Application No. 10/088,708 is a 371 of international application No. PCT/US00/25595, filed on Sep. 19, 2000.
(63) Continuation of application No. 09/400,147, filed on Sep. 21, 1999, now abandoned.

(51) Int. Cl. A61K 31/473

(52) U.S. Cl. 514/290

(57) ABSTRACT

The use of desloratadine for the preparation of a medicament for treating and/or preventing allergic and inflammatory conditions of the skin or upper and lower airway passages in a human while avoiding the food effect associated with non-sedating antihistamines, e.g., loratadine or fexofenadine is disclosed.

### BACKGROUND OF THE INVENTION

This invention relates to treating and/or preventing allergic and inflammatory conditions in a human while avoiding a food effect associated with non-sedating antihistamines by administering an amount of desloratadine effective for such treating and/or preventing.

Loratadine is disclosed in U.S. Pat. No. 4,282,233 as a non-sedating antihistamine useful for treating allergic reactions in animals including humans. Fexofenadine is disclosed in U.S. Pat. Nos. 4,254,129 and 5,275,693 as a non-sedating antihistamine useful for treating allergic disorders.

Food intake induces changes in the physiology of the gastrointestinal tract that may influence drug absorption and/or drug clearance. Physiological changes induced by food intake can result in, inter alia , delayed gastric emptying, stimulation of bile flow, changes in pH, and increase in splanchnic blood flow. Food intake can also alter luminal metabolism and physically or chemically interact with a drug substance. The effects of co-administration of meals with drugs is generally maximal when the drug product is administered immediately after completion of a meal. Meals that are high in calories, fat and density are likely to provide the greatest effects on bioavailability. While loratadine is safe and efficacious, there is a food effect associated with its administration. After co-administration of meals with loratadine, the effects of loratadine are higher than when loratadine is administered to a patient under fasted condition [See Physican's Desk Reference (PDR), 54^{th} Edition, 2000, Medical Economics Co., Montvale, N.J., at page 2782]. Fexofenadine has an opposite food effect; the effects of fexofenadine are greater when administered to a patient under fasted conditions (See CPS, 33^{rd} Edition, 1998 Canadian Pharmacists Association, Ottawa, Canada, at page 57).

In the case of another orally-active antihistamine, cetirizine hydrochloride, food had no effect on the cetirizine exposure (AUC), but the Tmax was delayed by 1.7 hours and the Cmax was decreased by 23% in the presence of food (See PDR, 54^{th} Edition, 2000, at page 2404).

Thus, the complex effects of food, and the physicochemical properties and formulation of antihistamines often make the effect of food intake upon the bioavailability of antihistamines unpredictable.

Desloratadine is disclosed in U.S. Pat. No. 4,659,716 as a non-sedating antihistamine useful for treating allergic reactions in animals including humans. We are aware of no publication regarding the association of food intake upon administration of desloratadine.

There is a need for a clinically effective therapy to treat or prevent such allergic and inflammatory conditions of the skin and airway passages in a human while avoiding the food effect associated with non-sedating antihistamines, such as loratadine and fexofenadine as well as other antihistamines such as cetirizine hydrochloride.

### SUMMARY OF THE INVENTION

The present invention provides a method of treating and/or preventing allergic and inflammatory conditions of the skin or airway passages in a human in need of such treating and/or preventing while avoiding a food effect associated with non-sedating antihistamines which comprises administering an amount of desloratadine effective for such treating and/or preventing.

The present invention also provides a method of treating and/or preventing seasonal or perennial allergic rhinitis in a human while avoiding a food effect associated with non-sedating antihistamines which comprises administering an amount of desloratadine effective for such treating and/or preventing.

The present invention provides a method of treating and/or preventing atopic dermatitis or urticaria in a human in need of such while avoiding a food effect associated with non-sedating antihistamines which comprises administering an amount of desloratadine effective for such treating and/or preventing.

### DETAILED DESCRIPTION OF INVENTION

The phrase“allergic and inflammatory conditions of the skin or airway passages” is meant those allergic and inflammatory conditions and symptoms found on the skin and in the upper and lower airway passages from the nose to the lungs. Typical allergic and inflammatory conditions of the skin or upper and lower airway passages include seasonal and perennial allergic rhinitis, non-allergic rhinitis, asthma including allergic and non-allergic asthma, sinusitis, colds [in combination with a NSAID, e.g., aspirin or ibuprofen, or acetaminophen (APAP) and/or a decongestant, e.g., pseudoephedrine), dermatitis, especially allergic and atopic dermatitis, and urticaria and symptomatic dermographism as well as retinophathy, and small vessel diseases, associated with diabetes mellitus.

The amount of desloratadine effective for treating or preventing allergic and inflammatory conditions of the skin or airway passages will vary with the age, sex, body weight and severity of the allergic and inflammatory condition of the patient. Typically, the amount of desloratadine effective for treating or preventing such allergic and inflammatory conditions is in the range of about 2.5 mg/day to about 45 mg/day, preferably about 2.5 mg/day to about 20 mg/day, or about 5.0 mg/day to about 15 mg/day, or about 5.0 mg/day to about 10 mg/day, more preferably about 5.0 mg/day to about 7.5 mg/day, and most preferably about 5.0 mg/day in single or divided doses, or a single dose of 5.0 mg/day.

Desloratadine is a non-sedating long acting histamine antagonist with potent selective peripheral H1-receptor antagonist activity. Following oral administration, loratadine is rapidly metabolized to descarboethoxyloratadine or desloratadine, a pharmacologically active metabolite. In vitro and in vivo animal pharmacology studies have been conducted to assess various pharmacodynamic effects of desloratadine and loratadine. In assessing antihistamine activity in mice (comparison of ED₅₀ value), desloratadine was relatively free of producing alterations in behavior alterations in behavior, neurologic or autonomic function. The potential for desloratadine or loratadine to occupy brain H1-receptors was assessed in guinea pigs following i.p. administration and results suggest poor access to central histamine receptors for desloratadine or loratadine. In vivo studies also suggest that an inhibitory effect of desloratadine on allergic bronchospasm and cough can also be expected.

The clinical efficacy and safety of desloratadine has been documented in over 3,200 seasonal allergic rhinitis patients in 4 double-blinded, randomized clinical trials. The results of these clinical studies demonstrated the efficacy of desloratadine in the treatment of adult and adolescent patients with seasonal rhinitis.

Efficacy endpoints in all the studies were Total Symptom Score, Total Nasal Symptom Score, Total Non-nasal Symptom Score, and Health Quality of Life (HQOL) analysis in efficacy trials. Desloratadine (5 mg once daily) significantly reduced the total symptom scores (the sum of individual scores for rhinorrhea, sneezing, congestion/stuffiness, nasal itching, itchy/burning eyes, tearing, ocular redness, and itchy ears/palate). Desloratadine (5 mg) was significantly (p<0.01) more effective than placebo in reducing nasal symptoms. An important efficacy endpoint analyzed in the desloratadine studies is the AM NOW total symptom score. This parameter measures the total symptom relief by the patient after 24 hours before taking the next day dose. Statistically significant (p<0.05) reductions were maintained for the full 24 hour dosing interval over the entire 5 mg to 20 mg dosage range There were no significant differences in the effectiveness of desloratadine (over the entire 5 mg to 20 mg dosage range) across subgroups of patients defined by gender, age, or race. Desloratadine is particularly useful for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal allergic rhinitis, including nasal congestion, in patients in need of such treating and/or preventing.

Desloratadine is contraindicated in patients who are hypersensitive to this medication or to any of its ingredients.

### CLINICAL DESIGN FOR STUDY No. 1

This open-label, two-way crossover study in 18 healthly subjects was designed to evaluate the effect of food on the bioavailability of Desloratadine in accordance with the.FDA guidelines for evaluating the effect of food on the bioavailability of drugs. Subjects were randomized to receive single desloratadine 7.5 mg tablet under fasted conditions in one treatment period and immediately following a standardized high-fat, high-caloric breakfast meal in the other treatment period.

STUDY OBJECTIVE

The objective of this study was to evaluate the effect of food on the bioavailability of desloratadine.

INVESTIGATIONAL PLAN

Overall Study Design and Plan: Description

A total of 18 healthy subjects were enrolled and successfully completed this randomized, open-label, single-dose, two-way crossover study.

Subjects were screened within 3 weeks of dosing, and those who met the entry criteria were confined to the study center within 12 hours prior to each treatment (Day -1). Upon confinement, subjects had safety laboratory tests and electrocardiograms repeated. The following morning, after fasting for a minimum of 10 hours, subjects received one of the following treatments based on his/her subject number and the study period:

Treatment A: One desloratadine 7.5 mg tablet administered after a 10 hour fast.

Treatment B: One desloratadine 7.5 mg tablet administered immediately following a standardized high-fat high-caloric breakfast.

Subjects randomized to receive the standardized high-fat, high-caloric breakfast (Treatment B) consumed the prescribed meal in a 20-minute period prior to drug administration and received the appropriate dose of desloratadine within 5 minutes after completing the breakfast.

Each dose was administered with 180 mL (6 fl oz) of non-carbonated room temperature water. The tablet was swallowed whole, not chewed or crushed. After dosing, the oral cavity was inspected to assure that the subject had swallowed the tablet. Subjects continued fasting (Treatment A) or did nor eat again (Treatment B) until the 4-hr study procedures were completed, at which time lunch was served. Water was permitted throughout the fasting period except for 2 hours following treatment administration. The subjects remained awake and seated upright/ambulatory for 4 hours post-dose.

All subjects were confined to the study site until the 168-hour study related procedures were obtained. No strenuous physical activity was permitted, and the subjects were not allowed visitors while they were confined to the study site. A physician was present for all drug administrations and remained on the study site for at least four hours post-dose. A wash-out period of at least 7 days separated each period of the study.

Vital signs and ECGs were performed, and blood samples were collected at pre-specified times for safety and pharmacokinetic evaluations. Subjects were continually observed and questions throughout the study for possible occurrence of adverse events. Subjects were also instructed to report any unusual experiences or discomfort.

Overall Study Design: Discussion Since this study was conducted to determine the influence of food on the oral bioavailability (AUC and Cmax) of desloratadine, using the fasting state as reference, an open-label, randomized, two-way crossover design was used to meet the study objective.

Study Population/Inclusion Criteria/Exclusion Criteria

Inclusion Criteria:

Subjects were males or females between the ages of 18 and 45 years inclusive, and had a Body Mass Index (BMI) between 19-27.

Clinical laboratory tests (CBC, blood chemistries, urinalysis) were within normal limits or clinically acceptable to the Investigator/Sponsor.

Drug screen for drugs with a high potential for abuse were negative at screening and on admission to the study site.

Subjects were free of any clinically significant disease that required a physician's care and/or may have interfered with study evaluations, procedures or participation.

Subject gave written informed consent (prior to any study-related procedures being performed) and were willing to adhere to restrictions and examination schedules.

Subjects had a normal or clinically acceptable physical examination and ECG.

Exclusion Criteria:

Subjects who had a history of any clinically significant local or systemic infectious disease within four weeks prior to initial treatment administration.

Subjects who did not comply with the requirement that he or she should not have used any drugs (except acetaminophen) within 14 days prior to the study nor alcohol or xanthine-containing substances with 72 hours prior to study drug administration.

Subjects who had participated in a clinical trial of any investigational drug within 30 days prior to the start of the study.

Subjects who were, or were known to be former, narcotic addicts or alcoholics.

Subjects who were positive for hepatitis B surface antigen or hepatitis C antibody.

Subjects who were positive for HIV antibodies.

Subjects who had a clinically significant history of food or drug allergy.

Subjects who had a known allergy or intolerance to loratadine.

Subjects who had donated blood within the preceding 30 days.

Subjects who smoked, used tobacco products or used an adjunct to smoking cessation within the past 6 months (positive urine continue test).

Females who were not surgically sterilized or were considering reversal of their surgical sterilization or were not at least 1 year post-menopausal.

Females who had a positive urine pregnancy test at screening or on admission to the study site.

Females who were lactating.

Study Treatments

Subjects were confined to the study site at least 12 hours prior to each treatment administration. In the morning of Day 1 following a 10 hour overnight fast, each subject received one of the following treatments based on his/her subject number and the study period. The order of treatment administration was determined according to a computer-generated random code supplied to the Investigator by the Sponsor:

Treatment A: One desloratadine 7.5 mg tablet adminstered after a 10 hour fast.

Treatment B: One desloratadine 7.5 mg tablet administered immediately following a standardized high-fat, high-caloric breakfast.

Subjects randomized to receive the standardized high-fat, high-caloric breakfast (Treatment B) consumed the prescribed meal in a 20-minute period prior to drug administration and received desloratadine 7.5 mg tablet within 5 minutes after completing the breakfast.

Each dose was administered with 180 mL (6 fl oz) of non-carbonated room temperature water. The tablet was swallowed whole, not chewed or crushed. After dosing, the oral cavity was inspected to assure that the subject had swallowed the tablet. Subjects continued fasting (Treatment A) or did not eat again (Treatment B) until the 4-hour study procedures were completed, at which time lunch was served. Water was permitted throughout the fasting period except for 2 hours following treatment administration. The subjects remained awake and seated upright/ambulatory for 4 hours post-dose.. Subjects were under medical supervision throughout their confinement at the study site. Each treatment administration was separated by at least a 7 day washout period.

The desloratadine tablets were manufactured, packaged and supplied to the Investigator by Schering Corporation, Kenilworth, N.J., USA.

Method of Treatment Assignment

This was a randomized, open-label, two-way crossover study. Upon confinement at the research center and after fulfilling all the study entry requirements, subjects were randomly assigned to received Treatment A (fasted condition) or Treatment B (fed condition) in one of the following two treatment-sequences according to a computer-generated random code supplied by Schering-Plough Research Institute.

AB

Or

BA

Subjects who withdrew or were removed from the study were to be replaced at the discretion of the Sponsor. Their replacement was to be numbered by using the original subject's assigned number plus an“R” (i.e., 1 replaced to 1R). The dosing regimen of the new subject was to be according to the original subject's dosing regimen.

Selection of Doses & Selection and Timing of Dose for Each Subject

The desloratadine dose of 7.5 mg was selected because this was projected to be the clinical dose; similar response is expected with other doses, e.g., 5 or 10 mg/day.

Each subject received a single 7.5 mg desloratadine tablet on two occasions.

Prior and Concomitant Therapy

No other medications (investigational, prescription or OTC) except acetaminophen were taken by the subjects within 14 days of treatment initiation or during the course of the study without prior approval from the Principal Investigator or Sponsor unless it was a medical emergency. The use of any medications, including analgesics and over-the-counter medications that may have been used to treat adverse events, were to be recorded on the appropriate page of the case record form.

Pharmacokinetics

Blood samples were collected for determination of the plasma pharmacokinetic profile of desloratadine. Fifteen milliliters (15 mL) of blood were collected just prior to drug administration (0 hour) and at 0.5, 1, 1.5, 2, 3, 4, 6, 8, 12, 24, 36, 48, 72, 96, 120, 144 and 168 hours after dosing in both periods. All blood samples were collected into heparin-containing tubes at the specified times.

The blood samples were centrifuged within 30 minutes after collection for 20 minutes at approximately 4° C. and at approximately 3000 rpm. The plasma was separated and transferred into two separate appropriately labeled tubes, frozen to at least −20° C. and maintained in the frozen state until assayed for desloratadine content.

The plasma concentration data for desloratadine (following administration of desloratadine 7.5 mg) was used to estimate the following pharmacokinetic parameters:

Cmax —maximum observed plasma concentration

Tmax —time of observed maximum plasma concentration

AUC(tf) area under the plasma concentration vs time curve from time zero to the final measurable sampling time(tf)

AUC(l) —area under the plasma concentration vs time curve from time zero to infinity(l)

K —terminal phase rate constant

The major pharmacokinetic variables of interest were the plasma AUC(tf) and Cmax. All plasma samples were assayed for desloratadine concentration using a validated GLC/NPD method. The validation of the assay methods included documentation of its selectivity, limit of quantitation, linearity, precision and accuracy. The lower limit of quantitation (LOQ) of the assay was established at 0.1 ng/mL for desloratadine.

Plasma desloratadine concentration-time data were used to determine the pharmacokinetic parameters using model-independent methods. The maximum plasma concentration (Cmax) and time of maximum plasma concentration (Tmax) were the observed values. The terminal phase rate constant (K) was calculated as the negative of the slope of the log-linear terminal portion of the plasma concentration-time curve using linear regression. The terminal phase half-life (t1/2) was calculated as 0.693/K.

The area under the plasma concentration-time curve from time zero to the time of the final quantifiable sample [AUC(tf)] was calculated using the linear trapezoidal method and was extrapolated to infinity (I) according to the following equation:

AUC(I)=AUC(tf)+C(tf)K

where C(tf) is the estimated concentration at tf.

Safety Measurements Assessed

For safety evaluation, physical examinations, vital signs, electrocardiograms and clinical laboratory tests were conducted at screening and at the conclusion of the study (168 hours post-treatment). In addition, vital signs were monitored prior to treatment administration and daily during both treatment periods. Additional clinical laboratory tests and ECGs were obtained prior to dosing in each treatment period. The assessment, severity and relationship to treatment of adverse events were evaluated .

SUMMARY CONCLUSIONS FOR Study No. 1:

RESULTS: Study No. 1 was conducted as planned.

Clinical Pharmacology: The pharmacokinetics for the two-way cross-over study are presented in Tables I and II hereinbelow.

TABLE I
The Mean (% CV) pharmacokinetic parameters of desloratadine
following oral, single-dose administration of 7.5 mg under fed and
fasted conditions:

| | | FED | FASTED |
|---|---|---|---|
| Parameter (Unit) | N | Mean (% CV)¹ | Mean (% CV) |
| Cmax(ng/mL) | 18 | 3.53 (33) | 3.30 (36) |
| AUC(tf)(ng · hr/mL) | 18 | 73.8 (81) | 77.5 (92) |
| AUC(l)(ng · hr/mL) | 17 | 62.5 (40) | 63.5 (45) |

¹% CV is percent coefficient of variation, which is a relative measure of variability. See Steele and Torrie, “Principles and Procedures of Statistics”, (1980) 2^{nd} Edition, McGraw-Hill, NY, at page 27.

TABLE II
The estimates of bioavailability (log-transformed) of desloratadine
under fed condition relative to that after fasting

| Parameter | Point Estimate(%)^{c} | 90% Confidence Interval^{d} |
|---|---|---|
| Cmax(ng/mL)^{a} | 108 | 99-118 |
| AUC(tf)(ng · hr/mL)^{a} | 100 | 93-107 |
| AUC(l)(ng · hr/mL)^{b} | 101 | 93-108 |

^{a}n = 18
^{b}n = 17
^{c}Ratio of the mean value for fed vs. fasted.
^{d}α = 0.05(two-tailed)

Safety: Blood pressure, pulse rate, respiratory rate, oral body temperature and electrocardiogram evaluations showed no consistent changes of clinical relevance and remained within the range observed for healthy subjects. Overall, 9 of 18 (50%) subjects reported treatment-emergent adverse events. The most frequently reported adverse event was headache. All reported adverse events were mild in severity except one such event that was reported as moderated. No subject discontinued participation in the study due to adverse events and no intervention was required to treat any adverse event.

Conclusions for Study No. 1:

Single oral does of desloratadine 7.5 mg administered under fed and fasted conditions were safe and well tolerated.

Food intake had no effect on the oral bioavailability of desloratadine from the tablet formulation.

### CLINICAL DESIGN FOR STUDY No. 2

This open-label, three-way crossover study in 30 healthly subjects was designed to evaluate the effect of food on the bioavailability of desloratadine syrup in accordance with the FDA guidelines for evaluating the effect of food on the bioavailability of drugs and to determine the bioequivalence of desloratadine between the tablet and syrup formulations. Subjects were randomized to receive one desloratadine 5.0 mg tablet under fasted conditions in a one treatment period or ten(10) mL of desloratadine syrup (0.5 mg/mL) under fasted conditions in a second treatment period or ten(10) mL of desloratadine syrup(0.5 mg/mL) immediately following a standardized high-fat, high-caloric breakfast meal in third treatment period.

Study Objective

The objectives of Study No. 2 were to determine the bioequivalency of desloratadine between the tablet and syrup formulations and to evaluate the effect of food on the bioavailability of desloratadine following administration of a syrup formulation.

Overall Study Design and Plan:

A total of 30 healthy subjects were enrolled and successfully completed this randomized, open-label, single-dose, three-way crossover study.

Subjects were screened within 3 weeks of dosing, and those who met the entry criteria were confined to the study center within 12 hours prior to each treatment (Day-1). Upon confinement, subjects had safety laboratory tests and electrocardiograms repeated. The following morning, after fasting for a minimum of 10 hours, subjects received one of the following treatments based on his/her subject number and the study period:

| | |
|---|---|
| Treatment A: | One desloratadine 5 mg tablet administered |
| | after a 10-hour fast. |
| Treatment B: | Ten (10) mL of desloratadine syrup |
| | (0.5 mg/mL) following a 10-hour fast. |
| Treatment C: | Ten (10) mL of desloratadine syrup |
| | (0.5 mg/mL) administered immediately |
| | following a standardized high-fat and high- |
| | caloric breakfast. |

Subjects who were randomized to receive the standardized high-fat and high-caloric breakfast (Treatment C), consumed the prescribed meal in a 20-minute period prior to drug administration and received the appropriate dose of desloratadine within 5 minutes after completing the breakfast.

The tablets were administered with 180 mL (6 fl oz) of noncarbonated room temperature water. The tablet was swallowed whole, not chewed or crushed. After dosing, the oral cavity was inspected to assure that the subject swallowed the tablet/syrup. For subjects randomized to Treatment B or Treatment C the study medication was administered by having the volunteer drink the entire 10 mL of desloratadine syrup, followed by two 10 mL tap water rinses of the dose container (ie, oral syringe, etc.) to ensure complete dose intake. Subjects consumed the remaining 160 mL of water. Subjects continued fasting (Treatments A and B) or did not eat again (Treatment C) until the 4-hour study procedures were completed, at which time lunch was served. Water was permitted throughout the fasting period except for 2 hours following treatment. The subjects remained awake and seated upright/ambulatory for 4 hours post-dose.

All subjects were confined to the study site until the 120-hour study related procedures were obtained. No strenuous physical activity was permitted, and the subjects were not allowed visitors while they were confined to the study site. A physician was present for all drug administrations and remained on the study site for at least four hours post-dose. A washout period of at least 14 days separated each period of the study.

Vital signs and ECGs were performed and blood samples were collected at prespecified times for safety and pharmacokinetic evaluations. Subjects were continually observed and questioned throughout the study for possible occurrence of adverse events. Subjects were also instructed to report any unusual experiences or discomfort.

Overall Study Design:

Since Study No. 2 was conducted to determine the bioequivalency of desloratadine between the tablet and syrup formulations and to evaluate the influence of food on the oral bioavailability (AUC and Cmax) of desloratadine of the syrup formulation an open-label, randomized, three-way crossover design was used to meet the study objective.

The Inclusion Criteria and Exclusion Criteria used for Study No. 2 were the same as those used for Study No. 1 except that:

Subjects who smoked, used tobacco products or used an adjunct to smoking cessation within the past 6 months (positive urine continue test) were not excluded; and

Females who were not post-menopausal or those who were not practicing an adequate contraceptive method as well as females who had a positive urine pregnancy test at screening or on admission to the study site, or those who were lactating were excluded.

Method of Treatment Assignment

This was a randomized, open-label, three-way crossover study. Upon confinement at the research center and after fulfilling all the study entry requirements, subjects were randomly assigned to receive Treatment A, Treatment B or Treatment C in one of the following six treatment-sequences according to a computer-generated random code:

| | |
|---|---|
| ABC | BCA |
| ACB | CAB |
| BAC | CBA |

Pharmacokinetics

Plasma concentrations of desloratadine and 3-OH desloratadine were determined using a validated liquid chromatography with tandem mass spectrometric (LC/MS/MS) method with a lower limit of quantitation (LOQ) of 0.025 ng/mL and a linear range of 0.025-10 ng/mL for each analyte.

The mean and %CV were calculated for plasma concentrations of desloratadine and 3-OH desloratadine at each time point. Concentration values less than the assay LOQ (0.025 ng/mL) were reported as and set to zero in the calculations. The plasma concentration-time data for desloratadine and 3-OH desloratadine were then subjected to pharmacokinetic analysis by noncompartmental methods using the WinNonlin™ Professional computer program. For each subject, the following pharmacokinetic parameters were determined: maximum plasma concentration (Cmax), time of maximum plasma concentration (Tmax), area under the plasma concentration-time curve (AUC), time of final quantifiable sample (tf) and terminal phase half-life (t ½). Cmax, Tmax and ff were the observed values. The AUC values from time zero to the final measurable sampling (AUC[tf]) and from time zero to infinity (AUC[I]) were calculated using the linear trapezoidal method described for Study No. 1.

The terminal phase rate constant (K) was calculated as the negative of the slope of the log-linear terminal portion of the plasma concentration-time curve using linear regression. The t ½ was calculated as 0.693/K.

Pharmacokinetic Assessment of Desloratadine.

The mean plasma concentration-time data for desloratadine and 3-OH desloratadine are summarized in Tables III and IV.

Following oral administration, desloratadine was absorbed and slowly metabolized to 3-OH desloratadine (the active metabolite). In general, the absorption of desloratadine from either tablet (fasted, Treatment A) or syrup (fasted [Treatment B] and fed [Treatment C]) occurred rapidly with no lag time.

The mean pharmacokinetic parameters for desloratadine among treatments were similar, ranging from 2.19 to 2.44 ng/mL for Cmax, and 47.4 to 52.0 ng-hr/mL for AUC(I), (Table III). For 3-OH desloratadine, similar results were observed (Table III). The mean values for Cmax, and AUC(I), ranged from 0.91-1.06 ng/mL, and 27.8-29.0 ng-hr/mL, respectively.

Statistical comparisons of log-transformed Cmax and AUC(I) values for Treatment B versus Treatment A and Treatment C versus Treatment B were performed for both desloratadine and 3-OH desloratadine Overall, the results suggested that there were no statistically significant differences in the bioavailabilities of desloratadine from the tablet and syrup formulations, and that high-fat and high-caloric meals had no effect on the bioavailability of desloratadine from the syrup formulation.

The relative bioavailability and the 90% Confidence Inteverals (CIs) for the log-transformed Cmax and AUC(I) for desloratadine and 3-OH desloratadine are presented in Table IV. The CIs of AUC(I) and Cmax of desloratadine and 3-OH desloratadine for Treatment B relative to Treatment A met the 80-125% bioequivalence guideline. This indicates that the tablet and syrup formulations were bioequivalent. The corresponding CIs for the syrup under fasted and fed conditions met the bioequivalency criteria for Cmax (70-143%) and AUC (80-125%). Thus, a high-fat and high-caloric meal had no effect on the bioavailability of desloratadine or 3-OH desloratadine from the syrup formulation. There was also no effect of food previously seen with the tablet formulation.

TABLE III
Mean (% CV) Pharmacokinetic Parameters of
Desloratadine and 3-OH Desloratadine in Healthy
Adult Volunteers Following Single-Dose Oral
Administration of Desloratadine Tablet and Syrup
Formulations Under Either Fasted or Fed Condition
(N = 30)
Pharmacokinetic Parameters

| | Cmax | AUC(tf) | AUC(l) |
|---|---|---|---|
| Treatment | (ng/mL) | (ng · hr/mL) | (ng · hr/mL) |
| | Desloratadine | | |
| A | 2.44 (41) | 45.8 (44) | 47.4 (45) |
| B | 2.30 (51) | 46.2 (71) | 48.4 (74) |
| C | 2.19 (62) | 49.9 (90) | 52.0 (93) |
| | 3-OH Desloratadine | | |
| A | 1.06 (34) | 27.0 (25) | 29.0 (24) |
| B | 1.03 (38) | 26.0 (28) | 27.8 (28) |
| C | 0.91 (38) | 25.8 (31) | 28.3 (31) |

Where:
Treatment A = One 5 mg desloratadine tablet administered after a 10-hour fast.
Treatment B = Ten (10) mL desloratadine syrup (0.5 mg/mL) administered after a 10-hour fast.
Treatment C = Ten (10) mL desloratadine syrup (0.5 mg/mL) administered immediately following a standardized high-fat and high-caloric breakfast.

TABLE IV
Estimates of Bioequivalence and the 90% Confidence Intervals
for the Log-Transformed Cmax and AUC(l) for Desloratadine
and 3-OH Desloratadine in Healthy Adult Volunteers Following
Single-Dose Oral Administration of Desloratadine Tablet and
Syrup Formulations Under Fasted or Fed Condition

| | | |
|---|---|---|
| | Relative | Confidence |
| Formulations Compared | Bioavailability (%) | Interval (%)^{a} |

Desloratadine

| | | | |
|---|---|---|---|
| Treatment B/ | AUC(l) | 95.4 | 84-108 |
| Treatment A | Cmax | 92.5 | 84-102 |
| Treatment C/ | AUC(l) | 104 | 92-118 |
| Treatment B | Cmax | 94.1 | 85-104 |

3-OH Desloratadine

| | | | |
|---|---|---|---|
| Treatment B/ | AUC(l) | 94.9 | 89-101 |
| Treatment A | Cmax | 96.5 | 89-104 |
| Treatment C/ | AUC(l) | 101 | 95-108 |
| Treatment B | Cmax | 87.2 | 81-94 |

^{a}Ninety percent confidence interval.
Where:
Treatment A = One 5 mg desloratadine tablet administered after a 10-hour fast.
Treatment B = Ten (10) mL desloratadine syrup (0.5 mg/mL) administered after a 10-hour fast.
Treatment C = Ten (10) mL desloratadine syrup (0.5 mg/mL) administered immediately following a standardized high-fat, high-caloric breakfast.

SAFETY: Overall, 14 of 30 subjects (47%) reported at least one adverse event (“AE”) during the study. Eight of 30 (27%) subjects reported at least one AE during the fasted treatment period with the tablet, 4 of 30 (13%) subjects reported at least one AE during the fasted treatment period with the syrup and 7 of 30 (23%) subjects reported at least one AE during the fed treatment period with the syrup. The most common AE (regardless of association to treatment) was headache.

Six and 5 subjects reported headaches of mild and moderate severity, respectively. All headaches rated as moderate were treated with acetaminophen.

Ten of 30 subjects (33%) AEs were considered to be treatment-related treatment-emergent AEs. The most common treatment-related AEs were headache (10 of 30; 33%) and gastrointestinal disorders (2/30; 7%).

No serious nor unexpected AEs were reported. No subject discontinued participation in the study due to AEs.

There were no deaths or serious or significant adverse events. There were no clinically significant abnormal laboratory values. Blood pressure, pulse rate, electrocardiograms and oral body temperature evaluations showed no consistent changes of clinical relevance and remained within the range observed for healthy male and female subjects.

Overall Conclusions for Study No 2:

The administration of desloratadine 5 mg, in either a tablet or syrup formulation, was found to be safe and well tolerated in this study. All adverse events were considered mild to moderate in severity and the incidence of adverse events was similar across treatments. No subject was discontinued from the study due to an adverse event.

In Study, the bioequivalence of the 5 mg desloratadine syrup formulation (0.5 mg/mL) was evaluated with the 5 mg desloratadine tablet formulation. Based on the 90% CIs for Cmax and AUC, the 2 formulations were found to be bioequivalent with respect to desloratadine as well as its major metabolite, 3-OH desloratadine. In addition, the relative bioavailability of the syrup under fed and fasted conditions was evaluated. No food effect was observed for the syrup as evidenced by the 90% CIs which also met the bioequivalence guidelines.

Conclusions for Study No. 2:

The tablet and syrup desloratadine formulations were bioequivalent.

High-fat and high-caloric meal had no effect on the bioavailability of desloratadine from the syrup formulation.

### CONCLUSIONS FOR STUDY NOs. 1 & 2:

Thus, food intake had no effect on the bioavailability of oral administrated desloratadine from the tablet or syrup formulations. Similar results are expected when the effect of food intake on the bioavailability of the rapidly-disintegrating desloratadine formulation is evaluated.

### GENERAL EXPERIMENTAL

U.S. Pat. No. 4,659,716 discloses methods of making desloratadine, pharmaceutical compositions containing it and methods of using desloratadine and pharmaceutical compositions containing it to treat allergic reaction in mammals.

U.S. Pat. No. 5,595,997 discloses pharmaceutical compositions containing desloratadine and methods of using desloratadine for treating and preventing various disease states, e.g., allergic rhinitis.

Desloratadine is available from Schering Corporation, Kenilworth, N.J.

The pharmaceutical compositions of desloratadine can be adapted for any mode of administration e.g., for oral, e.g. tablet, syrup or rapidly-disintegrating tablet, parenteral, e.g., subcutaneous (“SC”), intramuscular (“IM”), and intraperitoneal (“IP”), topical or vaginal administration or by inhalation (orally or intranasally). Preferably desloratadine is administered orally.

Such pharmaceutical compositions may be formulated by combining desloratadine or an equivalent amount of a pharmaceutically acceptable salt thereof with a suitable, inert, pharmaceutically acceptable carrier or diluent that may be either solid or liquid. Desloratadine may be converted into the pharmaceutically acceptable acid addition salts by admixing it with an equivalent amount of a pharmaceutically acceptable acid. Typically suitable pharmaceutically aceptable acids include the mineral acids, e.g., HNO₃, H₂SO₄, H₃PO₄, HCI, HBr, organic acids, including, but not limited to, acetic, trifluoroacetic, propionic, lactic, maleic, succinic, tartaric, glucuronic and citric acids as well as alkyl or arylsulfonic acids, such as p-toluenesulfonic acid, 2-naphthalenesulfonic acid, or methanesulfonic acid. The preferred pharmaceutically acceptable salts are trifluoroacetate, tosylate, mesylate, and chloride. Desloratadine is more stable as the free base than as an acid addition salt and the use of the desloratadine free base in pharmaceutical compositions of the present invention is more preferred.

Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Penn.

Liquid form preparations include solutions, suspensions and emulsions. As an example may be mentioned water or water-propylene glycol solutions for parenteral injection. Solid form preparations may be converted into liquid preparations shortly before use for either oral or administration. Parenteral forms to be injected intravenously, intramuscularly or subcutaneously are usually in the form of sterile solutions and may contain tonicity agents (salts or glucose), and buffers. Opacifiers may be included in oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active component, e.g., an effective amount to achieve the desired purpose.

What is claimed:

1) A method of treating or preventing allergic and inflammatory conditions of the skin or airway passages in a human in need of such treating or preventing while avoiding a food effect associated with other non-sedating antihistamines which comprises orally administering to said human under feed or fasted conditions an amount of desloratadine effective for such treating or preventing while avoiding a food effect on the bioavailability of desloratadine.

2) The method of claim 1 wherein the amount of desloratadine is about 2.5 mg/day to about 45 mg/day.

3) The method of claim 1 wherein the amount of desloratadine is about 2.5 mg/day.

4) The method of claim 1 wherein the amount of desloratadine is about 5 mg/day to about 10 mg/day.

5) The method of claim 1 wherein the amount of desloratadine is about 5 mg/day.

6) The method of claim 1 wherein the desloratadine is administered in a tablet formulation.

7) The method of claim 1 wherein the desloratadine is administered in a syrup formulation.

8) The method of claim 1 wherein the allergic reaction is season allergic rhinitis, pernninal allergic rhinitis, atopic dermatitis, urticaria or allergic asthma.

9) A method of treating or preventing allergic and inflammatory conditions of the skin or airway passages in a human in need of such treating or preventing a which comprises orally administering to said human an amount of desloratadine effective for such treating or preventing, while obtaining sustantially the same bioavailability of desloratadine under feed or fasted conditions.

10) The method of claim 9 wherein the amount of desloratadine is about 2.5 mg/day to about 45 mg/day.

11) The method of claim 9 wherein the amount of desloratadine is about 2.5 mg/day.

12) The method of claim 9 wherein the amount of desloratadine is about 5 mg/day to about 10 mg/day.

13) The method of claim 9 wherein the amount of desloratadine is about 5 mg/day.

14) The method of claim 9 wherein the desloratadine is administered in a tablet formulation.

15) The method of claim 9 wherein the desloratadine is administered in a syrup formulation.

16) The method of claim 9 wherein the allergic reaction is season allergic rhinitis, pernninal allergic rhinitis, atopic dermatitis, urticaria or allergic asthma.

17) A method of treating or preventing seasonal or perennial allergic rhinitis in a human in need of such treating or preventing while avoiding a food effect associated with non-sedating antihistamines which comprises orally administering to said human under feed or fasted conditions an amount of desloratadine effective for such treating or preventing while avoiding a food effect on the bioavailability of desloratadine.

18) The method of claim 17 wherein the amount of desloratadine is in the range of about 2.5 mg/day to about 45 mg/day.

19) The method of claim 17 wherein the amount of desloratadine is about 5 mg/day to about 15 mg/day.

20) The method of claim 17 wherein the amount of desloratadine is about 2.5 mg/day.

21) The method of claim 17 wherein the amount of desloratadine is about 5 mg/day.

22) The method of claim 17 wherein the patient is suffering from seasonal allergic rhinitis.

23) The method of claim 17 wherein the patient is suffering from perennial allergic rhinitis.

24) The method of claim 17 wherein the desloratadine is administered in a tablet formulation.

25) The method of claim 17 wherein the desloratadine is administered in a syrup formulation.

26) A method of treating or preventing seasonal or perennial allergic rhinitis in a human in need of such treating or preventing a which comprises orally administering to said human an amount of desloratadine effective for such treating or preventing, while obtaining sustantially the same bioavailability of desloratadine under feed or fasted conditions.

27) The method of claim 26 wherein the amount of desloratadine is in the range of about 2.5 mg/day to about 45 mg/day.

28) The method of claim 26 wherein the amount of desloratadine is about 5 mg/day to about 15 mg/day.

29) The method of claim 26 wherein the amount of desloratadine is about 2.5 mg/day.

30) The method of claim 26 wherein the amount of desloratadine is about 5 mg/day.

31) The method of claim 26 wherein the patient is suffering from seasonal allergic rhinitis.

32) The method of claim 26 wherein the patient is suffering from perennial allergic rhinitis.

33) The method of claim 26 wherein the desloratadine is administered in a tablet formulation.

34) The method of claim 26 wherein the desloratadine is administered in a syrup formulation.

35) A method of treating or preventing atopic dermatitis or urticaria in a human in need of such treating or preventing while avoiding a food effect associated with non-sedating antihistamines which comprises orally administering to said human under feed or fasted conditions an amount of desloratadine effective for such treating or preventing while avoiding a food effect on the bioavailability of desloratadine.

36) The method of claim 35 wherein the amount of desloratadine is about 2.5 mg/day.

37) The method of claim 35 wherein the amount of desloratadine is about 5 mg/day to about 15 mg/day.

38) The method of claim 35 wherein the amount of desloratadine is about 5 mg/day to about 10 mg/day.

39) The method of claim 35 wherein the amount of desloratadine is about 5 mg/day.

40) The method of claim 35 wherein the patient is suffering from atopic dermatitis.

41) The method of claim 35 wherein the patient is suffering from urticaria.

42) A method of treating or preventing atopic dermatitis or urticaria in a human in need of such treating or preventing a which comprises orally administering to said human an amount of desloratadine effective for such treating or preventing, while obtaining sustantially the same bioavailability of desloratadine under feed or fasted conditions.

43) The method of claim 42 wherein the amount of desloratadine is in the range of about 2.5 mg/day to about 45 mg/day.

44) The method of claim 42 wherein the amount of desloratadine is about 2.5 mg/day to about 45 mg/day.

45) The method of claim 42 wherein the amount of desloratadine is about 2.5 mg/day.

46) The method of claim 42 wherein the amount of desloratadine is about 5 mg/day to about 10 mg/day.

47) The method of claim 42 wherein the amount of desloratadine is about 5 mg/day.

48) The method of claim 42 wherein the desloratadine is administered in a tablet formulation.

49) The method of claim 42 wherein the desloratadine is administered in a syrup formulation.

50) The method of claim 42 wherein the patient is suffering from atopic dermatitis.

51) The method of claim 42 wherein the patient is suffering from urticaria.
